(19) United States
(12) Patent Application Publication
(10) Pub. No.: US 2019/0083128 A1
(43) Pub. Date: Mar. 21, 2019

(54) DECOMPRESSION NEEDLE AND METHOD FOR EMERENCY TREATMENT OF A PNEUMOTHORAX

(71) Applicant: H&H Medical Corporation, Williamsburg, VA (US)

(72) Inventor: Paul X. Harder, Williamsburg, VA (US)

(21) Appl. No.: 15/711,833

(22) Filed: Sep. 21, 2017

(51) Int. Cl. A61B 17/34; A61M 25/06

(52) U.S. Cl. CPC A61B 17/3415; A61M 1/04; A61M 25/0606

(57) ABSTRACT

A decompression needle including: a hollow catheter shaft including a distal end having an opening, a proximal end and a passage between the opening and proximal end; and an outer surface of or on the shaft, wherein at least a portion of the shaft proximate to the distal end has surface features configured to engage chest tissue to thereby resist unintended movement of the catheter shaft with respect to the chest tissue.

### TECHNICAL FIELD

The field of the invention is chest decompression needles, and particularly decompression needles for emergency treatment of a pneumothorax.

### BACKGROUND OF THE INVENTION

A pneumothorax, such as a tension pneumothorax, is an injury to the chest that allows air to escape the lung and collect in the pleural space between the chest wall and the lung. Air build-up in the pleural space can collapse a lung, and suppress the flow of blood to and from the heart. Gunshot wounds, impacts to the chest such as due to a vehicle crash, blasts from an explosion, and such event can cause a tension pneumothorax. Without immediate treatment, a patient can die from a pneumothorax.

Emergency treatment of a tension pneumothorax often involves inserting a needle into the chest, which is referred to as decompression needle. This treatment inserts a pointed tip of the decompression needle through the chest wall and into the pleural cavity. Air trapped in the pleural cavity enters the distal end of the needle, flows through the hollow shaft of the needle and vents to the atmosphere. The needle remains in the chest while the patient is transported to a hospital or other medical facility where the chest wound can be treated by physicians and other medical professionals.

Securing the needle to the chest is a problem. If the needle slides with respect to the chest, the distal end of the needle may move deeper into the pleural cavity and tear the lung. Or, the needle may slide out of the pleural cavity and not vent air from the cavity.

Monitoring the position of the needle in the chest is a task assigned to emergency medical technicians (EMTs), combat medics, medical evacuation flight crews and other emergency caregivers who are caring for the patient. This care is given in an emergency, which may involve combat or other dangerous environments. The caregivers may also be handling other tasks, such as attending to other injuries of the patient, moving the patient out of a dangerous environment and attending to other patients. Requiring the caregivers to monitor the position of a decompression needle in the patient is an added task that may or may not be properly performed by the caregiver in view of the dangerous environment and other tasks to be performed.

### SUMMARY OF INVENTION

A decompression needle configured for emergency treatment of tension pneumothorax has been invented and is disclosed herein. The needle includes an elongated hollow catheter shaft configured to be inserted through the chest wall such that a distal end of the shaft is positioned in a pleural cavity of a patient. An outer surface of the catheter shaft or a sheath covering the shaft has barbs, ridges, knurling or other surface features adapted to engage chest wall tissue. These surface features engage the chest tissue and thereby anchor the needle shaft to the chest.

Because the outer surface features on the catheter shaft anchor the chest tissue, the shaft does not move or moves only small distances with respect to the chest wall as the patient is treated and transported to a hospital or other medical facility. Because the catheter shaft does not substantially move, the distal end of the shaft remains positioned in the pleural cavity and air vents from the cavity through the shaft and into the atmosphere. The anchoring of the catheter shaft also relieves the emergency responders from having to continually monitor the position of the shaft in the chest and periodically reposition the catheter in the chest. Further, the anchoring reduces risks to the patient due to the distal end of the catheter shaft moving too deep into the pleural cavity and tearing the lung or that the distal end moving out of the cavity such that the needle no longer vents air trapped in the cavity.

An embodiment of the invention is a decompression needle including: a hollow catheter shaft including a distal end having an opening, a proximal end and a passage between the opening and proximal end; and an outer surface of or on the shaft, wherein at least a portion of the shaft proximate to the distal end has surface features configured to engage chest tissue to thereby resist unintended movement of the catheter shaft with respect to the chest tissue.

An embodiment of the invention is a decompression needle comprising: a hollow catheter shaft including a distal end having an opening, a proximal end and a passage between the opening and proximal end; and an outer surface of or on the shaft, wherein at least one anchor feature extends radially outward from a substrate region of the outer surface of the shaft.

### BRIEF DESCRIPTION OF THE DRAWINGS

FIG. 1 is schematic diagram showing a side view of an embodiment of the inventive needle.

FIG. 2 is a schematic cross-sectional diagram showing an enlarged view of a distal region of the needle shown in FIG. 2, wherein the needle is oriented in FIG. 2 at ninety (90) degrees about the axis of the needle as compared to the diagram of the needle shown in FIG. 1.

FIG. 3 is a schematic diagram showing the needle implanted in the chest wall.

FIG. 4 is a schematic diagram showing a side view of a distal portion of an alternative embodiment of the decompression needle.

FIG. 5 is a schematic diagram showing a side view and partially in cross section of a distal portion of an alternative embodiment of the decompression needle.

### DETAILED DESCRIPTION OF INVENTION

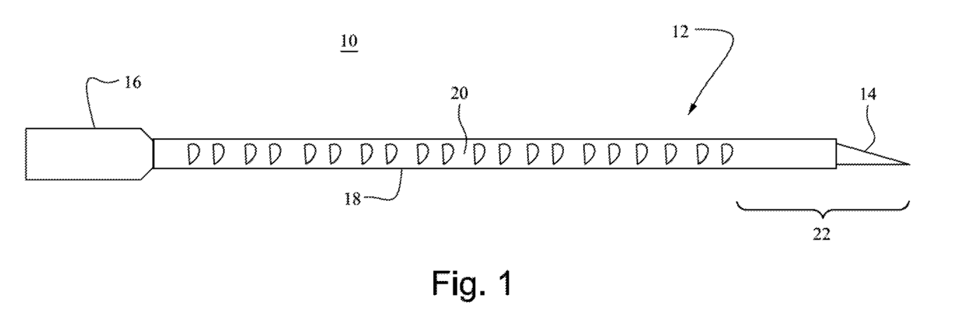

FIG. 1 shows a needle 10 configured for needle thoracostomy. The needle includes an elongated hollow catheter shaft 12 and a sharpened tip 14 at the distal end of the needle. The proximal end of the shaft 12 is connected to a vent 16 which may include a one way valve to allow air to escape the shaft but not enter the proximal end.

The outer surface 18 of the catheter shaft includes an engaging surface region 20 having surface features (also referred to as anchors) formed of barbs, ribs, scales, hairs or other surface features configured to anchor the catheter shaft to the tissue of the chest wall. These surface features may be configured to provide a certain level of resistance to the movement of the catheter shaft with respect to the chest wall. The surface features are also configure to allow the catheter shaft to be forcibly pushed through the chest wall and to allow the distal end 22 of the catheter shaft to be positioned in the pleural cavity and to resist. Further, the surface features are configured to allow the catheter shaft to be removed from the chest wall without inflicting substantial damage to the chest tissue. However, the surface features may unintentionally inflict some tissue damage as the catheter shaft is being removed from the chest.

The catheter shaft 12 may be a decompression needle having a diameter of 10 gauge, 14 gauge, 16 gauge, or other sized suitable for removing trapped air from the pleural cavity. The length of the catheter shaft may be a few inches, such as one to three inches, 6 to 7 cm, and 1.75 inch in length. The catheter shaft may be rigid shaft formed of a metal, such as stainless steel. Alternatively the catheter shaft may be a flexible tube such as a chest tube.

The distal end 22 of the catheter shaft may be sharpened to a pointed tip. The pointed tip is used to puncture the skin and push through the chest wall to the pleural cavity.

The catheter shaft 12 is hollow such that a lumen extends from the distal end 22 to the proximal end that couples to the vent 16. Air trapped in the pleural cavity enters the distal end, flows through the lumen and is discharged from the vent. The vent 16 may be a one-way valve, such as a flapper valve, that allows air to vent out from the catheter shaft and prevents air entering the shaft through the vent 16.

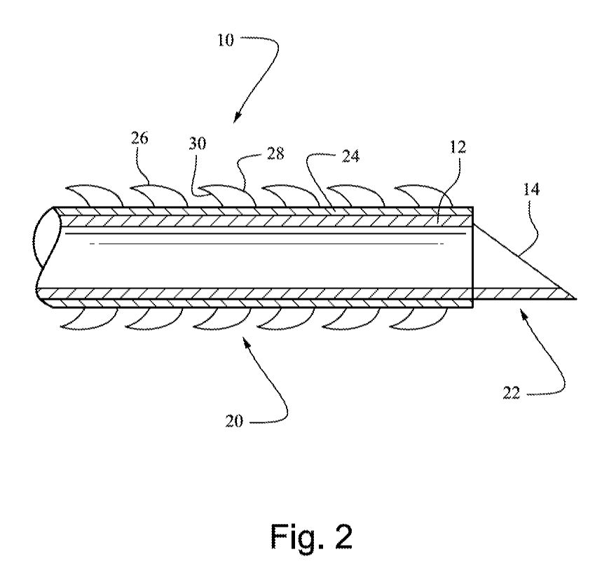

FIG. 2 shows in cross section a region of the catheter shaft 12 near the distal end 22. The catheter shaft 12 is shown as a rigid, metal shaft having a tip 14 formed by a diagonal cut at the distal end of the shaft.

A sheath 24 covers the catheter shaft and forms an outer surface of the needle 10. The sheath has a surface region 20 with barbs 26 extending radially outward from the sheath. The sheath may have a thickness of one-sixteenth to one-eighth of an inch. The sheath may extend the length of the catheter shaft or only a portion of the shaft near the distal end and intended to be inserted into chest tissue.

The barbs are each oriented with a rounded or otherwise smoothly changing surface 28 on a forward facing direction of the barb. The forward facing surfaces of the barbs tend not to grab the chest tissue as the catheter shaft is inserted into the chest tissue. The rearward facing portion of the barb 20 has a hook, overhang or other feature that is configured to engage the chest tissue and thereby resist movement of the catheter shaft out from the chest tissue.

The dimensions of the barb or other surface feature are such that the barb or surface feature engages the chest tissue to prevent inadvertent sliding of the catheter shaft while allowing the shaft to be intentionally removed (pulled out) from the chest wall without causing substantial injury to the tissue in the wall. The tissue may be irritated and abraded by the removal of the catheter shaft. The dimension of the barb or other surface feature may be a height above the surface of the catheter (in a radial direction) of, for example, one-sixteenth to one-eighth of an inch, a width (perpendicular to the axis of the catheter shaft) of one-sixteenth to one-eighth of an inch and a length (parallel to the axis) of one-sixteenth to one-quarter of an inch.

The sheath 24 and the barbs 26 may be formed of a deformable and flexible plastic material, such as a medical grade shape-memory polymer. The barbs 26 may be arranged in annular or helical rows on the surface of the sheath. Alternatively, the barbs may have an irregular arrangement on the surface of the sheath.

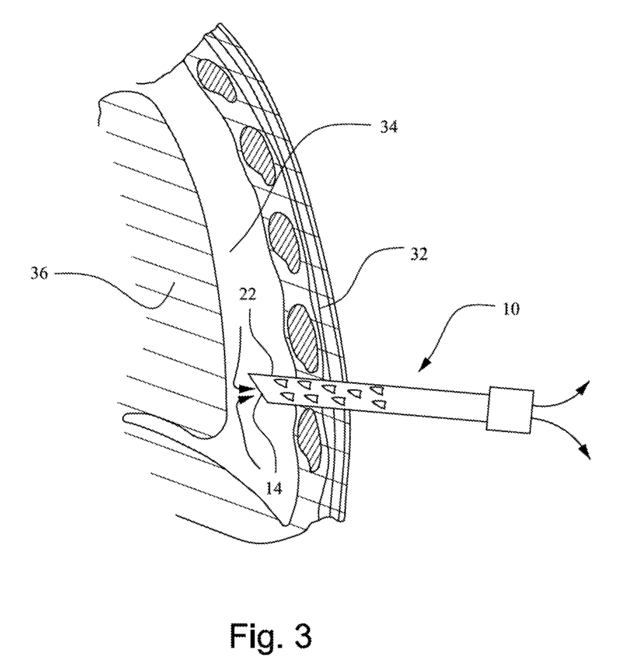

FIG. 3 shows an embodiment of the decompression needle 10 inserted in a chest wall 32. The distal end 22 of the needle is in the pleural cavity 34 of the patient's chest. Air (see arrows) in the pleural cavity enters the open end at the tip 14 of the needle. The air is exhausted through the vent 16 at the proximal end of the decompression needle 10. The venting of air from the pleural cavity allows air to escape the cavity 34. The venting of air allows the cavity to decompress which allows the lung 36 to inflate so that the patient may more easily breathe.

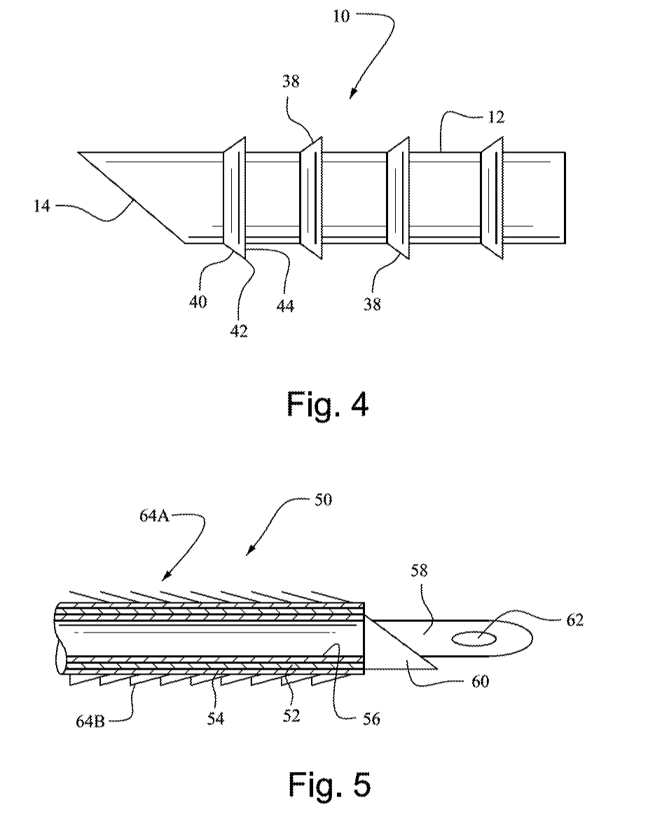

FIG. 4 shows an embodiment of the decompression needle 10 having ridges 38 formed directly on the metal outer surface of the catheter shaft 12. The ridges may be annular or helical with respect to the axis of the catheter shaft. The ridges may be oriented so that their forward facing surfaces 40 form a gradual incline from the surface of the catheter shaft to a ring rim 42. The back facing surface 44 may be perpendicular to the axis of the shaft or turn back inward to the ridge. The back facing surfaces 44 and the ring rim 42 of the ridges 38 are configured to engage the chest tissue and resist reward movement of the catheter shaft 10. Alternatively, the back facing surfaces 44 may have a similar slope but in an opposite direction to the forward facing surfaces 40, such that each ridge has an equilateral triangular cross sectional shape. Such ridges would resist equally sliding of the catheter shaft into and out of the chest wall.

FIG. 5 is a schematic diagram showing the distal region of an alternative compression needle 50 having a rigid and metallic catheter shaft 52, a sheath 54 and a ventilation tube 56. The ventilation tube slides through the hollow passage (lumen) of the catheter shaft. The distal end 58 of the ventilation tube is extended out from the tip 60 of the catheter shaft after the tip is positioned in the pleural cavity. By extending the distal end 58 out of the tip 60, the share end of the tip is partially shielded by the distal end. Thus, if the lung expands into the tip of the catheter shaft or the shaft slides towards the lung, the distal end will touch the lung before the catheter tip. The blunt end of the distal end 58 of the ventilation tube 56 is likely to do less damage to lung tissue than the damage done by the sharp tip 60 of the catheter shaft.

The distal end 58 of the ventilation tube has an opening 62 configured to receive air trapped in the pleural cavity. Air enters the opening 62, flows through the ventilation tube and is vented out a one way vent valve at the proximal end of the compression needle 50.

The sheath 52 has one or more surface features 64a, 64b which engage the chest tissue and thereby resist unintentional movement of the compression needle 50 with respect to the chest. The surface feature may be hairs 64a or scales 64b formed on the outer surface of the sheath. The scales or hairs may be oriented to resist movement outer of the chest wall, into the chest wall or both. It is not suggested that a single sheath have both scales and hairs, but such an embodiment may be desirable for certain applications.

The surface features on the outer surface of the catheter shaft (whether it be directly on the metal surface of the shaft or on a sheath covering the shaft) are configured to engage the chest tissue and thereby anchor the compression needle to the chest. The surface features may be the barbs, ridges, scales, and stiff hairs shown in FIGS. 1 to 5. Other surface features are suitable that similarly engage the chest tissue and aid in preventing unintended moving of the catheter shaft with respect to the chest.

The secure engagement between the needle and the chest is temporary and occurs only while the decompression needle is in the chest. The needle may be inserted in the chest for a few tens of minutes as the patient is moved to a hospital.

The needle is removed from the chest and the removal should not create significant damage to the tissue. Forming the barbs or other surface feature of a deformable material will allow the feature to deform while the catheter shaft is removed from the chest and thereby reduce or eliminate further injury to tissue permanent has barbs, is knurled or other surface features adapted to engage chest wall tissue.

While at least one exemplary embodiment of the present invention(s) is disclosed herein, it should be understood that modifications, substitutions and alternatives may be apparent to one of ordinary skill in the art and can be made without departing from the scope of this disclosure. This disclosure is intended to cover any adaptations or variations of the exemplary embodiment(s). In addition, in this disclosure, the terms “comprise” or “comprising” do not exclude other elements or steps, the terms “a” or “one” do not exclude a plural number, and the term “or” means either or both. Furthermore, characteristics or steps which have been described may also be used in combination with other characteristics or steps and in any order unless the disclosure or context suggests otherwise. This disclosure hereby incorporates by reference the complete disclosure of any patent or application from which it claims benefit or priority.

The invention is:

1. A decompression needle comprising:
a hollow catheter shaft including a distal end having an opening, a proximal end and a passage between the opening and proximal end; and
an outer surface of or on the shaft, wherein at least a portion of the shaft proximate to the distal end has surface features configured to engage chest tissue to thereby resist unintended movement of the catheter shaft with respect to the chest tissue.

2. The decompression needle of claim 1 wherein the surface features are at least one of barbs, ridges, hairs, scales and knurling.

3. The decompression needle of claim 1 wherein the surface features each have an abrupt change facing way from the distal end.

4. The decompression needle of claim 1 further comprising a sheath covering at least a portion of the catheter shaft and the outer surface is part of the sheath.

5. The decompression needle of claim 4 wherein the sheath is a shape memory polymer.

6. The decompression needle of claim 1 wherein the surface features are deformable.

7. The decompression needle of claim 1 wherein the surface features are formed of a shape memory polymer.

8. The decompression needle of claim 1 further comprising a ventilation tube in and coaxial to the catheter shaft and having a distal end of the ventilation tube is extendible from the distal end of the catheter shaft.

9. The decompression needle of claim 1 wherein the distal end of the catheter shaft includes a sharp end of the catheter shaft.

10. A decompression needle comprising:
a hollow catheter shaft including a distal end having an opening, a proximal end and a passage between the opening and proximal end; and
an outer surface of or on the shaft, wherein at least one anchor feature extends radially outward from a substrate region of the outer surface of the shaft.

11. The decompression needle of claim 10 wherein the anchor feature is a plurality of anchor features distributed over the outer surface on the shaft.

12. The decompression needle of claim 10 wherein the anchor feature is at least one of barbs, ridges, hairs, scales and knurling.

13. The decompression needle of claim 1 wherein the anchor feature has an abrupt change in a surface feature facing way from the distal end.

14. The decompression needle of claim 1 further comprising a sheath covering at least a portion of the catheter shaft and the anchor feature is part of the sheath.

15. The decompression needle of claim 14 wherein the sheath is a shape memory polymer.

16. The decompression needle of claim 10 wherein the anchor feature is deformable.

17. The decompression needle of claim 10 wherein the anchor feature is formed of a shape memory polymer.

18. The decompression needle of claim 10 further comprising a ventilation tube in and coaxial to the catheter shaft and having a distal end of the ventilation tube is extendible from the distal end of the catheter shaft.

19. The decompression needle of claim 10 wherein the distal end of the catheter shaft includes a sharp end of the catheter shaft.

20. The decompression needle of claim 10 further comprising a one-way ventilation valve at the proximal end of the catheter shaft.
